(19) United States
(12) Patent Application Publication
(10) Pub. No.: US 2026/0269074 A1
(43) Pub. Date: Sep. 10, 2026

(54) CANCER PROGNOSIS RISK ASSESSMENT METHOD, SYSTEM AND COMPUTER PROGRAM PRODUCT

(71) Applicant: FU JEN CATHOLIC UNIVERSITY, New Taipei (TW)

(72) Inventors: Chen-Wei YU, Taipei (TW); Rui-Bin LIN, Hsinchu (TW)

(21) Appl. No.: 19/550,636

(22) Filed: Feb. 26, 2026

Related U.S. Application Data
(60) Provisional application No. 63/766,481, filed on Mar. 4, 2025.

(51) Int. Cl. G16H 50/30

(52) U.S. Cl. CPC G16H 50/30

(57) ABSTRACT

A method for cancer prognosis risk assessment is provided. The method includes: receiving gene expression data of a target subject and reference gene expression data of multiple reference samples; selecting a target immune gene and constructing a pseudo-time series by ranking observed values of the reference samples and positioning the target subject among the multiple reference samples; performing a fitting operation on the pseudo-time series with a time-series statistical model to generate a target fitted value; calculating a residual value based on a difference between a target observed value and the target fitted value; and inputting the residual value and expression values of the multiple immune gene signatures into a deep learning model to generate a prognosis risk score of the target subject. A system and a computer program product using the method are also provided.

### CROSS-REFERENCE TO RELATED APPLICATION(S)

The present disclosure claims the benefit of U.S. Provisional Application No. 63/766,481 filed on Mar. 4, 2025. The entire disclosure of the above-mentioned prior application is hereby incorporated by reference herein

### FIELD

The present disclosure is generally related to the field of bioinformatics and medical data analysis, and more specifically, to a method, a system, and a computer program product for cancer prognosis risk assessment.

### BACKGROUND

In current bioinformatics technologies, cancer prognosis assessment mostly relies on direct statistical analysis or machine learning prediction using gene expression levels. However, gene expression data from a single time point or in a static form often contains substantial linear trends and background noises, making it difficult for models to capture subtle and representative heterogeneous features that are associated with changes in the tumor microenvironment.

In addition, although some studies have attempted to process bioinformatics data using deep learning models, most of these models lack effective feature engineering for high-dimensional and highly correlated gene signatures, resulting in insufficient clinical significance of model prediction results. Therefore, how to exclude general regularities in gene expression data and effectively integrate immune microenvironment features to accurately estimate the survival risk of cancer patients remains an urgent problem to be solved in the fields of clinical medicine and bioinformatics.

### SUMMARY

In view of the above, the present disclosure provides a cancer prognosis risk assessment method, system, and computer program product that construct a pseudo-time series and perform de-trending on a target immune gene through a time-series statistical model to extract residual data, and combine immune feature data for deep learning computation, thereby effectively addressing the problem in the prior art where static gene expression data contains substantial linear trends and background noise, making it difficult for models to capture heterogeneous fluctuations in the tumor microenvironment and resulting in insufficient accuracy and clinical significance of prognosis assessment. Through the technical means of the present disclosure, non-linear interaction features between the target gene and the immune environment can be extracted, thereby accurately generating a prognosis risk score and providing a reference with high statistical significance for clinical medical decision-making.

A first aspect of the present disclosure provides a method for cancer prognosis risk assessment, adapted for assessing a prognosis risk of a target subject, including: receiving gene expression data of the target subject and reference gene expression data of multiple reference samples; selecting a target immune gene from the gene expression data and the reference gene expression data; obtaining a target observed value of the target immune gene from the gene expression data, and obtaining multiple reference observed values of the target immune gene from the reference gene expression data; ranking the multiple reference samples based on magnitudes of the multiple reference observed values, and positioning the target subject among the multiple reference samples based on a magnitude of the target observed value, so as to construct a pseudo-time series; performing a fitting operation on the pseudo-time series with a time-series statistical model to generate a target fitted value corresponding to a position of the target subject in the pseudo-time series; calculating a difference between the target observed value and the target fitted value to generate a residual value; selecting multiple immune gene signatures from the gene expression data; and inputting the residual value and expression values of the multiple immune gene signatures into a deep learning model to generate a prognosis risk score of the target subject.

In some implementations of the first aspect, the time-series statistical model is an Autoregressive Integrated Moving Average (ARIMA) model.

In some implementations of the first aspect, inputting the residual value into the deep learning model includes: converting the residual value into a tensor format corresponding to an input format of the deep learning model.

In some implementations of the first aspect, the multiple immune gene signatures includes at least one of CD8 T cells, PRF1, GZMB, or Th1.

In some implementations of the first aspect, selecting the multiple immune gene signatures includes: selecting multiple candidate immune gene signatures from the gene expression data; calculating correlation coefficients among expression values of the multiple candidate immune gene signatures; performing a cluster analysis on the multiple candidate immune gene signatures based on the correlation coefficients; and selecting the multiple immune gene signatures based on a result of the cluster analysis.

In some implementations of the first aspect, the correlation coefficients are Spearman correlation coefficients.

A second aspect of the present disclosure provides a cancer prognosis risk assessment system adapted for assessing a prognosis risk of a target subject, including: a memory storing at least one computer-executable instruction; and a processor coupled to the memory. When the processor executes the at least one computer-executable instruction, the system is caused to: receive gene expression data of the target subject, and reference gene expression data of multiple reference samples; select a target immune gene from the gene expression data and the reference gene expression data; obtain a target observed value of the target immune gene from the gene expression data, and obtain multiple reference observed values of the target immune gene from the reference gene expression data; rank the multiple reference samples based on magnitudes of the multiple reference observed values, and position the target subject among the multiple reference samples based on a magnitude of the target observed value, so as to construct a pseudo-time series; perform a fitting operation on the pseudo-time series with a time-series statistical model to generate a target fitted value corresponding to a position of the target subject in the pseudo-time series; calculate a difference between the target observed value and the target fitted value to generate a residual value; select multiple immune gene signatures from the gene expression data; and input the residual value and expression values of the multiple immune gene signatures into a deep learning model to generate a prognosis risk score of the target subject.

In some implementations of the second aspect, the time-series statistical model is an Autoregressive Integrated Moving Average (ARIMA) model.

In some implementations of the second aspect, the system converts the residual value into a tensor format corresponding to an input format of the deep learning model when inputting the residual value into the deep learning model.

In some implementations of the second aspect, the multiple immune gene signatures include at least one of CD8 T cells, PRF1, GZMB, or Th1.

In some implementations of the second aspect, in selecting the multiple immune gene signatures, the at least one computer-executable instruction, when executed by the processor, further causes the system to: select multiple candidate immune gene signatures from the gene expression data; calculate correlation coefficients among expression values of the multiple candidate immune gene signatures; perform a cluster analysis on the multiple candidate immune gene signatures based on the correlation coefficients; and select the multiple immune gene signatures based on a result of the cluster analysis.

In some implementations of the second aspect, the correlation coefficients are Spearman correlation coefficients.

A third aspect of the present disclosure provides a non-transitory computer-readable medium storing at least one computer-executable instruction. When the at least one computer-executable instruction is executed by a processor of an electronic device, the electronic device is caused to: receive gene expression data of a target subject, and reference gene expression data of multiple reference samples; select a target immune gene from the gene expression data and the reference gene expression data; obtain a target observed value of the target immune gene from the gene expression data, and obtain multiple reference observed values of the target immune gene from the reference gene expression data; rank the multiple reference samples based on magnitudes of the multiple reference observed values, and position the target subject among the multiple reference samples based on a magnitude of the target observed value, so as to construct a pseudo-time series; perform a fitting operation on the pseudo-time series with a time-series statistical model to generate a target fitted value corresponding to a position of the target subject in the pseudo-time series; calculate a difference between the target observed value and the target fitted value to generate a residual value; select multiple immune gene signatures from the gene expression data; and input the residual value and expression values of the multiple immune gene signatures into a deep learning model to generate a prognosis risk score of the target subject.

In some implementations of the third aspect, the time-series statistical model is an Autoregressive Integrated Moving Average (ARIMA) model.

In some implementations of the third aspect, the electronic device converts the residual value into a tensor format corresponding to an input format of the deep learning model when inputting the residual value into the deep learning model.

In some implementations of the third aspect, the multiple immune gene signatures include at least one of CD8 T cells, PRF1, GZMB, or Th1.

In some implementations of the third aspect, in selecting the multiple immune gene signatures, the at least one computer-executable instruction, when executed by the processor, further causes the electronic device to: select multiple candidate immune gene signatures from the gene expression data; calculate correlation coefficients among expression values of the multiple candidate immune gene signatures; perform a cluster analysis on the multiple candidate immune gene signatures based on the correlation coefficients; and select the multiple immune gene signatures based on a result of the cluster analysis.

In some implementations of the third aspect, the correlation coefficients are Spearman correlation coefficients.

### BRIEF DESCRIPTION OF THE DRAWINGS

FIG. 1 is a flowchart of a cancer prognosis risk assessment method in accordance with an implementation of the present disclosure.

FIG. 2A is a Spearman correlation coefficient heatmap of immune gene signatures in accordance with an implementation of the present disclosure.

FIG. 2B is a hierarchical clustering dendrogram of immune gene signatures in accordance with an implementation of the present disclosure.

FIG. 3 is a block diagram of a computing system in accordance with an implementation of the present disclosure.

### DESCRIPTION

The following will refer to the relevant drawings to describe implementations of a cancer prognosis risk assessment method and system in the present disclosure, in which the same components will be identified by the same reference symbols.

The following description includes specific information regarding the exemplary implementations of the present disclosure. The accompanying detailed description and drawings of the present disclosure are intended to illustrate the exemplary implementations only. However, the present disclosure is not limited to these exemplary implementations. Those skilled in the art will appreciate that various modifications and alternative implementations of the present disclosure are possible. In addition, the drawings and examples in the present disclosure are generally not drawn to scale and do not correspond to actual relative sizes.

For consistency and ease of understanding, the same features are denoted by numerals in the exemplary drawings (although not always marked as such in some examples). However, features in different implementations may differ in other respects, and should not be narrowly confined to the features shown in the drawings.

Terms such as “at least one implementation,” “one implementation,” “various implementations,” “different implementations,” “some implementations,” “this implementation,” may indicate that the implementation(s) described as such may include specific features, structures, or characteristics, but not all possible implementations of the present disclosure need to include these specific features, structures, or characteristics. Moreover, the repeated use of the phrases “in one implementation,” “in this implementation” does not necessarily refer to the same implementation, although they may be. Furthermore, phrases like “implementation” used in conjunction with “the present disclosure” do not imply that all implementations must include specific features, structures, or characteristics, and should be understood to mean “at least some implementations of the present disclosure” include the specified features, structures, or characteristics. The term “coupled” is defined as a connection, whether direct or indirect, through an intermediate component, and is not necessarily limited to a physical connection. When the terms “comprising” or “including” are used, they mean “including but not limited to,” and explicitly indicate an open relationship between the combination, group, series, and the like.

Additionally, for the purpose of explanation and non-limitation, specific details such as functional entities, techniques, protocols, standards, etc., are set forth to provide an understanding of the described technology. In other examples, detailed descriptions of well-known methods, techniques, systems, architectures, etc., have been omitted to avoid unnecessarily obscuring the described implementations.

The terms “first,” “second,” and “third” and the like are used to distinguish different objects, not to describe a specific order. Furthermore, the terms “comprising” and any variations thereof are intended to cover non-exclusive inclusion. For example, a process, method, system, product, or device that includes a series of steps or modules is not limited to the listed steps or modules, but may optionally include unlisted steps or modules, or other steps or modules inherent to these processes, methods, products, or devices.

The cancer prognosis risk assessment technique provided by the present disclosure is a hybrid architecture combining a time-series statistical model and a deep learning model, and configured to process expression data of tumor-related immune genes, thus generating an accurate prognosis risk score.

Specifically, the implementation path of the present disclosure is to first perform de-trending on dynamically ranked gene data through a time-series statistical model (such as ARIMA) to extract residual data representing heterogeneous fluctuations, then fuse the residual data with a feature vector composed of specific immune gene signatures, and input the fused data into a deep learning model for training or inference computation. In some embodiments, the method described herein may be implemented in an assessment system having cloud computing capability. The system may include a data management module, a data processing module, and a cloud learning module that are executed by a processor configured to process high-dimensional biomedical big data.

In other words, the present disclosure does not simply use raw expression levels for prediction, but rather filters out general regular trends through a residual extraction technique, thus allowing more accurately captured subtle signals associated with cancer prognosis.

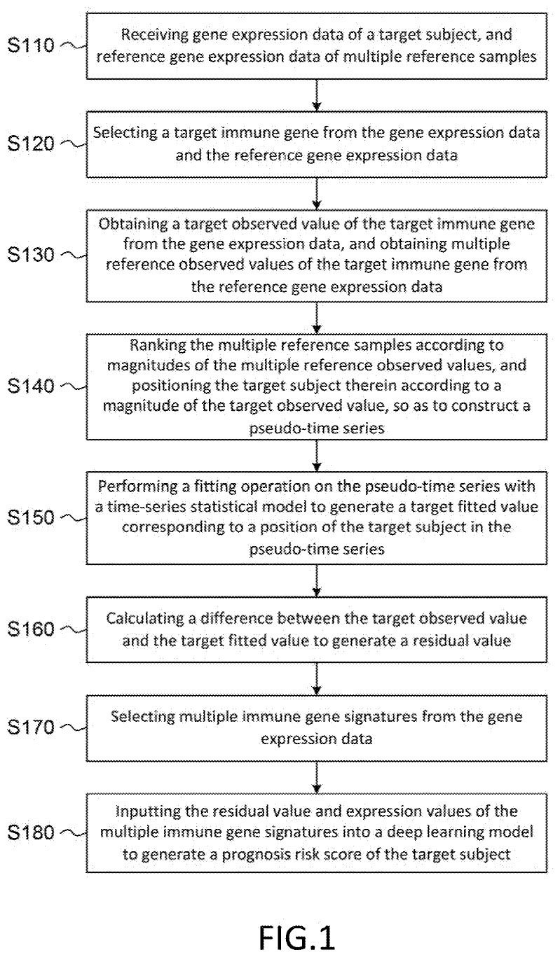

The implementation of the present disclosure will be described in a progressive manner herein. First, an overall flow of the cancer prognosis risk assessment method will be described with reference to FIG. 1. Then, a specific implementation of each action will be described in detail sequentially. In the course of description, a hepatocellular carcinoma dataset TCGA-LIHC (The Cancer Genome Atlas Liver Hepatocellular Carcinoma) will be used as an example to substantiate the technical effects of the present disclosure with concrete data. Finally, a system hardware architecture will be described.

FIG. 1 is a flowchart of a cancer prognosis risk assessment method in accordance with an implementation of the present disclosure.

Referring to FIG. 1, in action S110, gene expression data of a target subject and reference gene expression data of multiple reference samples are received. Specifically, the target subject is an individual whose prognosis risk is to be assessed, and gene expression measurement values of the target subject are generated from tumor tissue samples through RNA sequencing (RNA-seq), microarray, or other transcriptome quantification technologies. The multiple reference samples are a sample group used to establish a reference sequence. The multiple reference samples may be patient samples with known prognosis information, samples from public databases, or a previously established reference sample library. In some embodiments, the gene expression data is stored in a matrix form, in which rows of the matrix represent samples, columns of the matrix represent genes, and each element in the matrix represents an expression level of a corresponding gene in a corresponding sample.

In some implementations, the gene expression data may be RNA-seq normalized values, microarray signal values, or other transcriptome quantification results. In some implementations, for RNA-seq data, a system may receive normalized values in this action to eliminate batch effects and technical variations.

In some implementations, in terms of data sources, the gene expression data of the target subject and the reference gene expression data of the multiple reference samples may be uploaded by a user through a graphical interface, or may be imported from public database sources. For example, the user may upload a file in a CSV format or a TSV format, where the file includes sample identifiers, gene names, and corresponding expression level values. In some implementations, the system may connect to public databases (such as TCGA, GEO, ICGC, and the like), and the user may specify a dataset identifier or filtering criteria, so that the system automatically downloads and imports the reference gene expression data of the multiple reference samples. It should be noted that the term “system,” as used hereinafter, refers to a combination of hardware equipment and software modules configured to execute the aforementioned prognosis risk assessment method. Specifically, the system includes at least a processor and a memory coupled to the processor, and is capable of executing corresponding computational actions through the processor. The concept of the system is defined at this stage for ease of describing implementation of each method action in an automated computing environment. A specific hardware configuration and a detailed architectural diagram of the system will be disclosed in subsequent sections of the present specification. Through collaboration of the system, high-speed processing of massive biomedical data and automated risk prediction are advantageously achieved.

In some implementations, the implementation of this action is described using the TCGA-LIHC dataset as an example. The TCGA-LIHC dataset includes mRNA expression data and corresponding clinical survival data for 230 hepatocellular carcinoma samples. In an inference mode, the 230 samples may serve as the multiple reference samples for establishing a reference sequence for a target subject to be assessed. For example, when a new hepatocellular carcinoma patient (the target subject) needs to undergo prognosis risk assessment, the system receives the gene expression data of the target subject and uses the 230 samples from TCGA-LIHC as the multiple reference samples. In some implementations, the reference gene expression data of the multiple reference samples is organized into a matrix having a dimension of 230×N, where 230 represents the number of the multiple reference samples and N represents the number of genes. A person having ordinary skill in the art should understand that the value of N depends on a gene expression platform used and a data preprocessing method, and N may range from thousands to tens of thousands of genes.

In some implementations, in addition to receiving the gene expression data, this action may also receive clinical survival data corresponding to the multiple reference samples. The clinical survival data includes an overall survival time (OS_time) and an event indicator (event). Specifically, the overall survival time represents a length of time from diagnosis or treatment initiation to death or last follow-up, and is typically expressed in days, months, or years. The event indicator represents whether a death event has occurred for a patient corresponding to a sample. For example, event=1 indicates that the patient has died, and event=0 indicates that the patient is still alive.

In detail, the clinical survival data has different uses in different application scenarios. In a training mode, the clinical survival data is used to train the deep learning model, so that the deep learning model is enabled to learn an association between prognosis risk features and survival outcomes. Specifically, during training, the deep learning model generates a predicted risk score based on input features and uses the clinical survival data as a supervisory signal to optimize model parameters through a loss function (such as a Cox proportional hazards loss function or a survival probability loss function). In an inference mode, this action may not require the clinical survival data, but rather generates the prognosis risk score solely based on the gene expression data for assessing a prognosis risk of a new sample.

In other words, action S110 is a data input stage of the method of the present disclosure. In an inference mode, this action receives the gene expression data of the target subject to be assessed, and the reference gene expression data of the multiple reference samples, and optionally receives clinical survival data of the multiple reference samples, so as to provide fundamental data for subsequent time-series modeling and deep learning. Advantageously, by distinguishing the roles of the target subject and the multiple reference samples, the method of the present disclosure is able to utilize an established reference sample library to perform prognosis risk assessment on a new individual, and by receiving standardized and normalized gene expression data, accuracy and reproducibility of subsequent analysis are ensured, making the method applicable to gene expression data from different sources and different platforms.

Referring to FIG. 1, in action S120, a target immune gene is selected from the gene expression data and the reference gene expression data. The following provides a detailed description.

First, action S120 selects the target immune gene as an object of time-series modeling. Specifically, the target immune gene may be a gene associated with a tumor immune microenvironment, such as an immune chemokine, an immune checkpoint, a cytotoxicity-related gene, or another immune regulatory gene. The selection of the target immune gene may be based on biological prior knowledge, literature evidence, or a data-driven feature selection method. In some implementations, the target immune gene is selected as CCL5 (C-C Motif Chemokine Ligand 5). CCL5 is a chemokine that plays an important role in the tumor immune microenvironment and is capable of recruiting multiple types of immune cells (such as T cells, natural killer cells, and the like) to a tumor site.

In detail, the selection of the target immune gene may be performed in the following manners. In some implementations, a user may manually specify a name or an identifier of the target immune gene through a graphical interface. In some implementations, the system may provide a candidate immune gene list, and the user may select one or more target immune genes from the list. In some implementations, the system may automatically select an immune gene having a smallest p-value or a most significant hazard ratio as the target immune gene based on results of a univariate Cox regression analysis between genes and survival data. In some implementations, the system may select an immune gene having a larger variation based on a degree of gene expression variation (such as a standard deviation or a coefficient of variation) to ensure that the time-series modeling has a sufficient dynamic range.

In some implementations, the target immune gene may be any one of chemokine genes, such as CXCL9, CXCL10, CXCL11, CCL2, CCL3, CCL4, and CCL5. In some implementations, the target immune gene may be any one of immune checkpoint genes, such as PDCD1 (PD-1), CD274 (PD-L1), and CTLA4. In some implementations, the target immune gene may be any one of cytotoxicity-related genes, such as PRF1, GZMB, and GZMA. A person having ordinary skill in the art should understand that the selection of the target immune gene may vary depending on different cancer types, research purposes, or clinical questions.

In other words, action S120 establishes a gene target required for subsequent time-series modeling, laying a foundation for construction of the pseudo-time series and de-trending processing.

Referring to FIG. 1, in action S130, a target observed value of the target immune gene is obtained from the gene expression data, and multiple reference observed values of the target immune gene are obtained from the reference gene expression data. The following provides a detailed description.

Specifically, in action S130, the target observed value of the target immune gene is obtained from the gene expression data of the target subject received in action S110. Meanwhile, the multiple reference observed values of the target immune gene are obtained from the reference gene expression data of the multiple reference samples received in action S110. For example, assuming that the target subject is a new hepatocellular carcinoma patient and the target immune gene is CCL5, the target observed value is the expression level of the CCL5 gene in a tumor sample of the patient. If the multiple reference samples are the 230 samples from TCGA-LIHC, the multiple reference observed values are the respective CCL5 expression levels of the 230 reference samples, forming 230 reference observed values.

In some implementations, the manner of obtaining the observed values is to locate a corresponding column in the gene expression data matrix based on a name or an identifier of the target immune gene, and to obtain a value of each sample in the column. For example, assuming that a dimension of the gene expression data matrix is a number of samples× a number of genes and the target immune gene is CCL5, a column corresponding to CCL5 is extracted, in which an element corresponding to the target subject is the target observed value, and elements corresponding to the multiple reference samples are the multiple reference observed values.

In some implementations, the target observed value and the multiple reference observed values may be normalized values. In some implementations, if the target subject and the multiple reference samples are from different batches or different experimental conditions, the system may perform batch correction on the target observed value and the multiple reference observed values in this action to eliminate non-biological technical variations. For example, ComBat, Limma, or other batch correction methods may be employed, so that the target observed value and the multiple reference observed values are within a comparable numerical range.

In other words, action S130 obtains the target observed value of the target subject and the multiple reference observed values of the multiple reference samples for the target immune gene, providing a necessary foundation for subsequent construction of the pseudo-time series.

Referring to FIG. 1, in action S140, the multiple reference samples are ranked according to magnitudes of the multiple reference observed values, and the target subject is positioned therein according to a magnitude of the target observed value, so as to construct a pseudo-time series. The following provides a detailed description.

First, action S140 ranks the multiple reference samples according to the magnitudes of the multiple reference observed values. Specifically, assuming that the number of the multiple reference samples is N, where N is a natural number, the multiple reference samples are denoted as sample 1 to sample N, and the corresponding reference observed values are denoted as x₁, x₂, . . . , x_{N}. The multiple reference samples are ranked according to the magnitudes of the multiple reference observed values, for example, in ascending order, such that the ranked sequence of the multiple reference observed values satisfies x₁≤x₂≤ . . . ≤x_{N}. In some implementations, the ranking may be in descending order. After ranking, the multiple reference samples form a reference sequence based on the magnitudes of their respective reference observed values.

Next, action S140 positions the target subject at an appropriate position in the reference sequence according to the magnitude of the target observed value. Specifically, the target observed value of the target subject is denoted as x_{target}. The magnitude of x_{target} is compared with the magnitudes of the respective reference observed values in the reference sequence to find an appropriate insertion position, such that the sequence remains ordered after insertion. For example, assuming that the reference sequence is in ascending order, if x_{target} falls between xₖ and x_{{k+1}} (i.e., xₖ≤x_{target}<x_{{k+1}}), the target subject is inserted between sample k and sample k+1.

In some implementations, using the TCGA-LIHC dataset as an example, assuming that there are 230 reference samples, a reference sequence is formed by ranking the 230 reference samples in ascending order according to the reference observed values of CCL5. When a new hepatocellular carcinoma patient (the target subject) needs to be assessed, the CCL5 observed value (the target observed value) of the patient is obtained, and the patient is positioned at an appropriate position in the reference sequence formed by the 230 reference samples according to the magnitude of the target observed value. After positioning, a sequence including 231 elements is formed, in which 230 elements correspond to the multiple reference samples and 1 element corresponds to the target subject.

The sequence after the target subject is inserted is denoted as the pseudo-time series. Specifically, the pseudo-time series is an ordered sequence in which elements are arranged according to magnitudes of observed values of the target immune gene. A pseudo-time index is denoted as t, where t=1, 2, . . . , N+1 (N being the number of the multiple reference samples), and yₜ represents an observed value at position t of the pseudo-time series. One of the positions corresponds to the target subject, and the observed value at the position is the target observed value.

Those skilled in the art will understand that the concept of the pseudo-time series is derived from treating differences in observed values among samples as a dynamic change process. Specifically, although the samples may be obtained simultaneously in actual time, by ranking the samples according to the observed values of the target immune gene, the samples can be regarded as being at different biological states or disease progression stages. This ranking approach advantageously converts cross-sectional data into sequential data having a concept of a time axis, enabling the time-series statistical model to be applied for modeling a dynamic trend of the target immune gene.

In some implementations, the construction of the pseudo-time series may further consider other factors. For example, if the gene expression data includes covariates (such as age, gender, tumor stage, and the like), covariate adjustment may be performed on the observed values of the target immune gene before ranking to eliminate non-biological systematic differences. In some other implementations, the ranking may be performed based on joint observed values of multiple genes, for example, by calculating a composite score of samples through principal component analysis (PCA) or other dimensionality reduction methods, and performing ranking and positioning based on the composite score.

In other words, action S140 constructs a pseudo-time series including the target subject and the multiple reference samples by ranking the multiple reference samples and inserting the target subject, thus providing input data for subsequent time-series statistical model modeling.

Referring to FIG. 1, in action S150, a fitting operation is performed on the pseudo-time series with a time-series statistical model to generate a target fitted value corresponding to a position of the target subject in the pseudo-time series. The following provides a detailed description.

In action S150, the pseudo-time series formed in action S140 is modeled using the time-series statistical model. The time-series statistical model models the observed values of the pseudo-time series to capture a correlation structure and a trend among the observed values, and generates a sequence of fitted values. In some implementations, an Autoregressive Integrated Moving Average (ARIMA) model may be employed as the time-series statistical model. The ARIMA model is a time-series statistical model capable of capturing autocorrelation structures, trends, and random fluctuations in time-series data.

Specifically, the ARIMA model is defined by three parameters (p, d, q): p is an order of an autoregressive (AR) term, d is a number of differencing operations, and q is an order of a moving average (MA) term. A general form of the ARIMA (p, d, q) model may be expressed as:

$$ϕ ( B ) {( 1 - B )}^{d} {y}_{t} = θ ( B ) {ε}_{t}$$

where yₜ is an observed value at pseudo-time t, B is a backshift operator satisfying Byₜ=y_{{t-1}}, φ(B) is a p-th order autoregressive polynomial, θ(B) is a q-th order moving average polynomial, εₜ is a white noise residual term, and (1-B)^{d} represents a d-th order differencing operation.

In one implementation, the parameters of the ARIMA model are set to (5, 1, 0), i.e., p=5, d=1, and q=0. This parameter setting indicates that the model includes a 5th-order autoregressive term and a first-order differencing, and does not include a moving average term. Specifically, the ARIMA (5, 1, 0) model may be expressed as:

$$Δ {y}_{t} = c + {ϕ}_{1} Δ {y}_{\{ t - 1 \}} + {ϕ}_{2} Δ {y}_{\{ t - 2 \}} + {ϕ}_{3} Δ {y}_{\{ t - 3 \}} + {ϕ}_{4} Δ {y}_{\{ t - 4 \}} + {ϕ}_{5} Δ {y}_{\{ t - 5 \}} + {ε}_{t} ,$$

where Δyₜ=yₜ−y_{{t-1}} is a first-order difference, c is a constant term, φ₁ to φ₅ are autoregressive coefficients, and Et is a residual term.

In some implementations, the parameters (p, d, q) of the ARIMA model may be determined through an automatic selection method. For example, an Akaike Information Criterion (AIC) or a Bayesian Information Criterion (BIC) may be used as a model selection criterion to search for an optimal (p, d, q) combination within a candidate parameter range. In one implementation, the candidate parameter range is set to p=1 to 10, d=0 to 2, and q=0 to 10, all possible (p, d, q) combinations within this range are exhaustively evaluated, an AIC or BIC value is calculated for each combination, and a combination having a smallest AIC or BIC value is selected as a final model.

In some implementations, appropriate orders of p and q may be determined based on graphs of an autocorrelation function (ACF) and a partial autocorrelation function (PACF) of the sequence. Specifically, the ACF graph shows correlation coefficients between observed values at different time lags, and the PACF graph shows direct correlation coefficients after removing effects of intermediate time points. Those skilled in the art will understand that the ACF and PACF graphs may assist in determining appropriate orders of the autoregressive term and the moving average term. After model parameter estimation is completed, action S150 calculates a sequence of fitted values using the estimated model parameters. Specifically, for each position t in the pseudo-time series, a corresponding fitted value ŷₜ is calculated based on the ARIMA model formula and the estimated parameter values. The fitted value ŷₜ represents a prediction or estimation of the observed value yₜ by the ARIMA model.

Importantly, action S150 generates a fitted value corresponding to the position of the target subject in the pseudo-time series as the target fitted value. For example, assuming that the target subject is located at position t_{target} in the pseudo-time series, the target fitted value is ŷ_{{target}}. The target fitted value represents a prediction of the observed value of the target subject by the ARIMA model, reflecting an expected value of the target subject in the time-series trend.

In some implementations, other time-series statistical models may be employed in place of the ARIMA model. For example, an autoregressive (AR) model, a moving average (MA) model, a seasonal ARIMA (SARIMA) model, an exponential smoothing model, a vector autoregression (VAR) model, or other time-series models may be employed. In some other implementations, a trend decomposition method (such as STL decomposition) or a smoothing method (such as Loess smoothing or moving average smoothing) may be employed to extract a trend component of the sequence as the fitted values.

In other words, action S150 performs a fitting operation on the pseudo-time series with the time-series statistical model to capture a linear dynamic trend of the target immune gene in the sequence, and generates the target fitted value corresponding to the position of the target subject, thus providing a reference for subsequent calculation of the residual value.

Referring to FIG. 1, in action S160, a difference between the target observed value and the target fitted value is calculated to generate a residual value. The following provides a detailed description.

Specifically, action S160 calculates a difference between the target observed value of the target subject and the target fitted value generated in action S150. The target observed value is denoted as y_{target}, the target fitted value is denoted as ŷ_{target}, and the residual value ε_{target} is calculated as: ε_{target}=y_{target}−ŷ_{target}. The residual value represents a portion of the target observed value that cannot be explained by the time-series statistical model, reflecting a degree to which the target subject deviates from the overall trend in the expression of the target immune gene.

For example, assuming that the target subject is a new hepatocellular carcinoma patient, a CCL5 observed value (the target observed value) of the target subject is 5.2, and after fitting by the time-series statistical model, the target fitted value at the corresponding position is 4.8, the residual value is 5.2−4.8=0.4. A positive residual value indicates that the CCL5 expression of the target subject is higher than a trend value predicted by the time-series statistical model, and a negative residual value indicates that the CCL5 expression is lower than the trend value.

In the technical solution of the present disclosure, the residual value has a particular biological significance. Specifically, the time-series statistical model captures an overall linear trend of the target immune gene across the sample population, and the residual value reflects a degree to which an individual (the target subject) deviates from the population trend. This deviation may be associated with individual-specific immune microenvironment features, tumor heterogeneity, or other non-linear factors. Therefore, the residual value may serve as a feature having prognostic value and be input into the deep learning model for further analysis.

In some implementations, action S160 may also calculate residual values for all samples (including the multiple reference samples and the target subject) in the pseudo-time series. Specifically, for each position t in the pseudo-time series, a difference between the observed value yₜ at the position and the corresponding fitted value ŷₜ is calculated to generate a residual εₜ, where: εₜ=yₜ−ŷₜ.

In some implementations, for all positions t=1, 2, . . . , N+1 (where Nis the number of the multiple reference samples), a residual sequence {ε₁, ε₂, . . . , ε_{N+1}} may be generated. The residual sequence includes the residual values of all of the multiple reference samples and the target subject. In some implementations, in a training mode, the residual values of all samples may be calculated for training the deep learning model.

In other words, the residual value reflects a deviation between the observed value and a predicted value by the time-series statistical model. In a statistical sense, if the time-series statistical model is able to perfectly capture the linear dynamics and trend of the observed values, the residuals should approximate random white noise. However, in actual biological data, the residuals often contain non-linear patterns not captured by the linear model, interactions among immune genes, and other complex biological signals. These residual signals are precisely the key features to be extracted and learned by the subsequent deep learning model.

In some implementations, a standardization process may be performed on the residual value. For example, a mean and a standard deviation of the residual values of the multiple reference samples may be calculated, and the residual value of the target subject may be subtracted by the mean and then divided by the standard deviation to obtain a standardized residual value. The standardization process enables the residual value to have a zero mean and a unit variance, advantageously improving stability of subsequent processing by the deep learning model. In some other implementations, other transformations may be performed on the residual value, such as Min-Max normalization, Robust Scaling, or other normalization methods.

In other words, action S160 generates the residual value of the target subject by calculating the difference between the target observed value and the target fitted value, thus completing de-trending processing of the expression of the target immune gene. In some implementations, residual values of all samples may also be calculated for training or other analytical purposes. Advantageously, through the generation of the residual value, the method of the present disclosure decomposes the observed value of the target immune gene into two components: a trend component (the fitted value) explainable by the linear time-series model and a remaining signal (the residual value) not explainable by the linear model. This decomposition approach enables the subsequent deep learning model to focus on learning non-linear immune interactions and complex patterns contained in the residuals without interference from the linear trend, thus improving accuracy and robustness of the prognosis risk assessment.

Referring to FIG. 1, in action S170, multiple immune gene signatures are selected from the gene expression data. The following provides a detailed description.

In action S170, multiple immune gene signatures are selected from the gene expression data of the target subject received in action S110, and expression values thereof are extracted as features reflecting characteristics of the tumor immune microenvironment. The multiple immune gene signatures may include a single immune-related gene, an immune cell infiltration score, or a combined expression level of multiple immune genes. The expression values of the selected immune gene signatures will be input into the deep learning model together with the residual value generated in action S160, so as to extract non-linear interactions between the residual and immune features.

In detail, an immune gene signature is a set of genes associated with a specific immune cell type, an immune function, or an immune response. Each immune gene signature may include one or more genes, and expression levels thereof may reflect an infiltration degree of a corresponding immune cell or an activity of an immune function. For example, a CD8 T cells signature may include marker genes associated with CD8-positive T cells, and expression levels thereof may reflect an infiltration degree of CD8 T cells in tumor tissue. After action S170 selects the appropriate immune gene signatures, gene expression values corresponding to the immune gene signatures are extracted from the gene expression data of the target subject.

In some implementations, the selected immune gene signatures include at least one of CD8 T cells, PRF1, GZMB, and Th1, which are immune-related genes or gene sets. Specifically, a CD8 T cells signature may reflect an infiltration degree of cytotoxic T lymphocytes, PRF1 (Perforin 1) and GZMB (Granzyme B) are cytotoxicity-related genes, and a Th1 (Type 1 T helper cells) signature may reflect an activity of type 1 helper T cells. In some other implementations, the immune gene signatures may further include at least one of immune cell-related signatures such as B cells, Th2, T cells, NK cells (natural killer cells), Tregs (regulatory T cells), Macrophage, Granulocyte, and MDSCs (myeloid-derived suppressor cells).

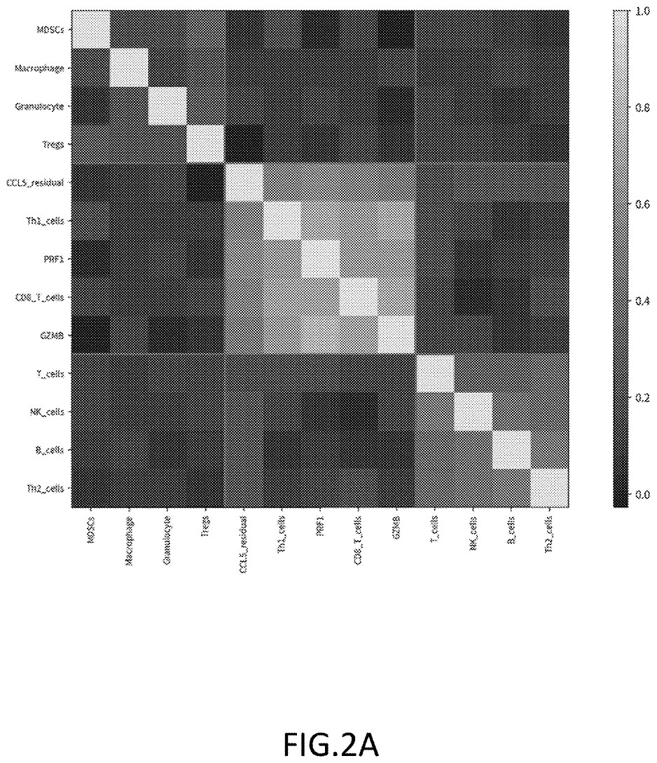

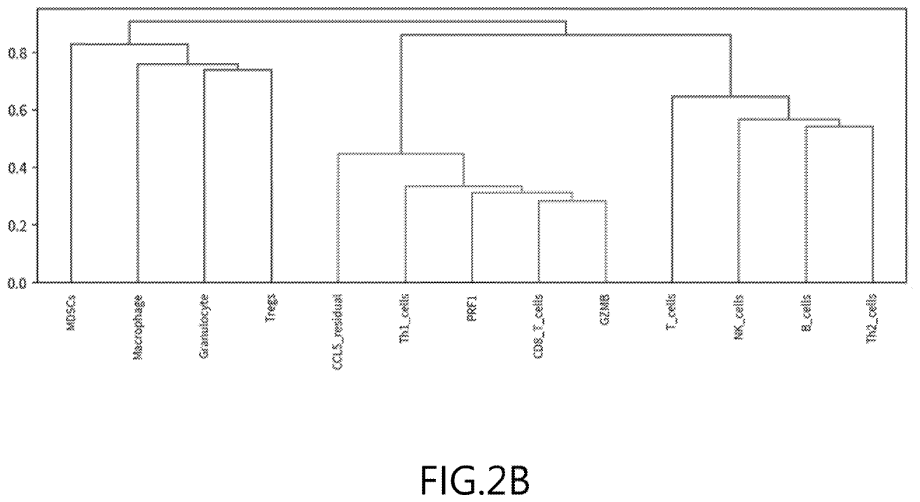

Referring to FIG. 2A, FIG. 2A is a Spearman correlation coefficient heatmap of immune gene signatures. The Spearman correlation coefficient heatmap presents correlations among the immune gene signatures in a matrix form. Specifically, a horizontal axis and a vertical axis of FIG. 2A respectively list names of the immune gene signatures, such as MDSCs, Macrophage, Granulocyte, Tregs, CCL5_residual, Th1_cells, PRF1, CD8_T_cells, GZMB, T_cells, NK_cells, B_cells, and Th2_cells. Each cell in the heatmap represents a Spearman correlation coefficient value between two corresponding immune gene signatures, and a shade of color represents a strength of the correlation.

In one implementation, the heatmap uses a color scale to indicate the correlation coefficients. For example, a dark color (such as dark blue or dark purple) represents a low correlation (with the correlation coefficient being close to 0), and a light color or a bright color (such as yellow) represents a high correlation (with the correlation coefficient being close to 1). A right side of FIG. 2A may include a color scale bar indicating a numerical range of the correlation coefficients, for example, from 0.0 to 1.0. Cells on a diagonal represent correlations of each immune gene signature with itself, the values of which are constantly 1, and are typically displayed in the brightest color.

By observing a heatmap pattern of FIG. 2A, groups of immune gene signatures having higher correlations may be identified. For example, if signatures such as CCL5_residual, Th1_cells, PRF1, CD8_T_cells, and GZMB form a lighter-colored block in the heatmap, this indicates that these signatures have higher positive correlations with one another, suggesting that they may jointly participate in cytotoxic immune responses. Similarly, if T_cells, NK_cells, B_cells, and Th2_cells form another block, this indicates that these signatures represent another group of related immune features.

Referring to FIG. 2B, FIG. 2B is a hierarchical clustering dendrogram of immune gene signatures. The hierarchical clustering dendrogram presents a clustering structure of the immune gene signatures. Specifically, a horizontal axis of FIG. 2B lists names of the immune gene signatures, and a vertical axis represents a clustering distance or dissimilarity. A distance matrix may be derived from a correlation coefficient matrix. For example, a distance d_{{j,k}}=1−ρ_{{j,k}}, where ρ_{{y,k}} is a Spearman correlation coefficient. The hierarchical clustering progressively merges similar signatures based on the distance matrix to form a tree structure (dendrogram).

In detail, the dendrogram of FIG. 2B extends from a bottom to a top. Leaf nodes at the bottom of the dendrogram correspond to respective immune gene signatures, branches connect similar signatures to form larger groups, and heights of the branches represent distance values at the time of merging. A smaller distance value indicates that two signatures or groups are more similar, and a larger distance value indicates a higher dissimilarity. The hierarchical clustering may employ different linkage methods, such as single linkage, complete linkage, average linkage, Ward's method, or other methods. In some implementations, FIG. 2B is labeled as “distance=1− Spearman rho,” indicating that the distance is defined as 1 minus the Spearman correlation coefficient.

In one implementation, as shown in FIG. 2B, the candidate immune gene signatures are divided into three panels through the hierarchical clustering analysis. A first panel may include myeloid-derived cell-related signatures such as MDSCs, Macrophage, Granulocyte, and Tregs, representing features associated with immunosuppression or innate immunity. A second panel may include cytotoxicity-related signatures such as CCL5_residual, Th1_cells, PRF1, CD8_T_cells, and GZMB, representing features associated with cytotoxic T cells and type 1 immune responses. A third panel may include other lymphocyte-related signatures such as T_cells, NK_cells, B_cells, and Th2_cells, representing diverse lymphocyte immune responses.

After the immune gene panels are formed, analysis may be performed separately based on different panels. Specifically, for each panel, expression data of the immune gene signatures included in the panel is extracted from the gene expression data. In some implementations, expression values of multiple immune gene signatures within a same panel may be combined, for example, by calculating a mean or a weighted mean, to generate a composite score for the panel. In some other implementations, independent expression values of respective immune gene signatures within the panel may be retained as a multidimensional feature vector, so as to preserve richer immune feature information.

In some implementations, normalization or standardization processing may be performed on the immune feature data. The normalization processing may eliminate scale differences among different immune gene signatures, advantageously improving training efficiency and feature extraction quality of the subsequent deep learning model.

In some implementations, other correlation coefficient calculation methods may be employed in place of the Spearman correlation coefficient. For example, a Pearson correlation coefficient, a Kendall's tau correlation coefficient, mutual information, or other similarity measurement methods may be used. In some other implementations, other clustering methods may be employed in place of the hierarchical clustering. For example, K-means clustering, DBSCAN, spectral clustering, or other clustering algorithms may be used. Those skilled in the art will understand that different correlation coefficients and clustering methods each have their own advantages and disadvantages, and the selection of an appropriate method may depend on data characteristics and analytical purposes.

In other words, through the Spearman correlation coefficient heatmap of FIG. 2A and the hierarchical clustering dendrogram of FIG. 2B, a correlation structure among the immune gene signatures may be visually presented, and the multiple immune gene signatures may be systematically organized into panels having biological significance. Each panel represents a group of functionally related immune features, advantageously assisting in selecting a representative and mutually independent combination of immune gene signatures, and improving biological interpretability and predictive value of the immune feature data.

Referring to FIG. 1, in action S180, the residual value and expression values of the multiple immune gene signatures are input into a deep learning model to generate a prognosis risk score of the target subject. The following provides a detailed description.

In action S180, the residual value generated in action S160 and the expression values of the multiple immune gene signatures obtained in action S170 are used as inputs and fed into the deep learning model for computation, so as to generate the prognosis risk score of the target subject. Specifically, the residual value represents a degree to which the target subject deviates from the population trend in the target immune gene (such as CCL5), the expression values of the immune gene signatures represent characteristics of the tumor immune microenvironment of the target subject, and the deep learning model generates a risk score capable of reflecting the prognosis risk by learning non-linear interactions between these two types of features.

In one implementation, before inputting the residual value and the expression values of the immune gene signatures into the deep learning model, action S180 first converts these data into a tensor format. Specifically, a tensor is a standard input format of the deep learning model and is typically a multidimensional array. The residual value and the expression values of the immune gene signatures need to undergo appropriate dimensional reshaping and combination to form a tensor conforming to input requirements of the deep learning model.

In a training mode, if multiple training samples need to be processed, the manner of tensor conversion is slightly different. Assuming that there are N training samples, each training sample corresponds to a residual value Et and an immune feature vector Sᵢ=[s_{{i,1}}, s_{{1,2}}, . . . , s_{{i,M}}}], where i=1, 2, . . . , N. The residual values and the immune feature vectors of all training samples may be combined into a three-dimensional tensor having a shape of (N, M+1, 1). Specifically, a first dimension is the number of training samples N, a second dimension is the number of features (M+1), and a third dimension is the number of channels 1.

In another implementation, a residual sequence may be processed in a manner of time window expansion. Specifically, in a training mode, if multiple training samples are ranked according to CCL5 observed values to form a pseudo-time series, a time window length w may be set, where w is a natural number. For each position t (t=w, w+1, . . . , N) in the pseudo-time series, w consecutive residual values {ε_{{t-w+1}}, ε_{{t-w+2}}, . . . , εₜ} preceding the position are extracted to form a time window having a length of w. This time window expansion manner may preserve local temporal dynamic information of the residual sequence, advantageously providing richer feature inputs for the deep learning model.

In some implementations, the deep learning model is a convolutional neural network (CNN). The CNN is a deep learning architecture that was originally widely applied in the fields of image recognition and computer vision, but has also been applied to biomedical data analysis in recent years. In the present disclosure, the CNN is used to extract local patterns and higher-order interactions between the residual value and the immune feature data, and to map these patterns to the prognosis risk score.

In detail, before inputting the residual value and the immune feature data into the CNN, action S180 may first convert these features into a tensor format suitable for input of the CNN. Specifically, assuming that the residual value is a scalar ε_{target} and the immune feature data is an M-dimensional vector S_{target}=[s₁, s₂, . . . , s_{M}], the residual value and the immune feature data may be concatenated into an (M+1)-dimensional feature vector X_{target}=[ε_{target}, s₁, s₂, . . . , s_{M}].

In some implementations, if the CNN expects a two-dimensional or three-dimensional tensor as input, dimensional expansion or reshaping may be performed on the feature vector. For example, the (M+1)-dimensional feature vector may be reshaped into a two-dimensional matrix or a three-dimensional tensor, such as arranging the feature vector into a 1×(M+1) matrix, or further expanding the feature vector into a 1×(M+1)×1 three-dimensional tensor. In some other implementations, a one-dimensional convolutional neural network (1D CNN) may be used to directly perform convolution operations on the feature vector.

An architecture of the CNN may include multiple layers for sequentially performing feature extraction and dimensionality reduction. In one implementation, the CNN includes at least one convolutional layer, at least one pooling layer, a flatten layer, at least one fully connected layer, and an output layer. Specifically, the convolutional layer extracts local features through convolution operations, the pooling layer performs downsampling to reduce dimensionality, the flatten layer flattens multidimensional features into a one-dimensional vector, the fully connected layer (also referred to as a dense layer) learns global associations among features, and the output layer generates a final prognosis risk score.

In some implementations, the convolutional layer may use a one-dimensional convolution (Conv1D) to perform convolution operations on the feature vector. A filter (also referred to as a kernel) slides across the feature vector to extract local patterns. The pooling layer may use max pooling or average pooling to select a maximum value or an average value within a local region as a representative value. In some implementations, multiple convolutional layers and pooling layers may be stacked to progressively extract higher-order feature representations.

In some implementations, the fully connected layer may include multiple hidden layers, each hidden layer including a number of neurons. The hidden layers may use a ReLU (Rectified Linear Unit) or other activation functions to introduce non-linearity. To prevent overfitting, a Dropout layer may be added between the fully connected layers to randomly discard outputs of a portion of neurons. The output layer typically includes one neuron and uses a linear activation function or a sigmoid activation function to generate the prognosis risk score.

In a training mode, the CNN is trained using the residual values, the immune feature data, and corresponding clinical survival data of multiple training samples. During training, the CNN generates predicted risk scores based on the input features and uses the clinical survival data as a supervisory signal to optimize model parameters through a loss function. In one implementation, the loss function may employ a Cox proportional hazards loss to accommodate right-censored data characteristics of survival analysis. In some other implementations, the loss function may employ a Mean Squared Error (MSE), a Cross-Entropy Loss, or other loss functions suitable for regression or classification tasks.

In an inference mode, the CNN uses trained model parameters to perform forward propagation on the residual value and the immune feature data of the target subject to generate the prognosis risk score of the target subject. The prognosis risk score is a continuous numerical value, where a higher value typically indicates a worse prognosis (high risk) and a lower value indicates a better prognosis (low risk). In some implementations, patients may be classified into a high-risk group and a low-risk group based on a median of the prognosis risk scores or other thresholds, so as to assist in clinical decision-making.

For example, assuming that the target subject is a new hepatocellular carcinoma patient, after processing through the aforementioned actions S110 to S170, a residual value ε_{target}=0.4 and an immune feature vector S_{target}=[2.1, 1.5, 0.8, 1.2] (assuming that four immune gene signatures are selected) are generated. Action S180 inputs these features into a trained CNN model, and after convolution, pooling, fully connected, and other operations, the CNN outputs a prognosis risk score of 0.65. The prognosis risk score may be compared with a distribution of prognosis risk scores of a reference population. If 0.65 is higher than a median, the patient is classified into a high-risk group, and more aggressive treatment or more intensive follow-up is recommended.

In other words, action S180 fuses the residual value and the immune feature data through the deep learning model (such as the CNN), learns complex non-linear interactions between the two, and generates a risk score capable of accurately reflecting the prognosis risk. Advantageously, by combining the de-trending residual from the time-series statistical model and the tumor immune microenvironment features, the method of the present disclosure is able to capture complex biological signals that cannot be processed by conventional linear models, significantly improving accuracy and clinical application value of the prognosis risk assessment.

In another implementation, a residual sequence may be processed in a manner of time window expansion. Specifically, in a training mode, if multiple training samples are ranked according to CCL5 observed values to form a pseudo-time series, a time window length w may be set, where w is a natural number. For each position t (t=w, w+1, . . . , N) in the pseudo-time series, w consecutive residual values {ε_{{t-w+1}}, ε_{{t-w+2}}, . . . , εₜ}

In some implementations, the deep learning model employs a multi-input architecture, in which the residual value and the immune feature data may serve as different input branches, respectively. For example, the residual value may serve as input branch 1, being a tensor having a shape of (1, 1, 1) or (N, 1, 1); and the immune feature data may serve as input branch 2, being a tensor having a shape of (1, M, 1) or (N, M, 1). The model internally processes the two input branches separately and then performs fusion at subsequent layers. In some other implementations, the residual and the immune features may be concatenated along a channel dimension to form a multi-channel tensor as a single input.

In one implementation, action S180 employs a convolutional neural network (CNN) as the deep learning model. In detail, a structure of the CNN includes an input layer, at least one convolutional layer, at least one pooling layer, a flatten layer, at least one fully connected layer (also referred to as a dense layer), and an output layer. Specifically, the input layer receives the tensorized residual value and expression values of the immune gene signatures, having a shape of, for example, (1, M+1, 1) representing a feature tensor of a single target subject, or (N, M+1, 1) representing a feature tensor of batch training samples.

The convolutional layer performs convolution operations on the input tensor, using multiple convolutional filters (also referred to as kernels) to extract local features. Each filter slides across the input tensor, calculates an inner product between the filter and a local region of the input, and generates a feature map. An activation function is typically applied following the convolutional layer to introduce non-linear characteristics. Commonly used activation functions include ReLU (Rectified Linear Unit), Leaky ReLU, ELU, or other non-linear functions.

In some implementations, a first convolutional layer of the CNN is a one-dimensional convolutional layer (Conv1D), in which a number of filters (also referred to as channels) may be set to 32, a kernel size may be set to 1, and an activation function may employ ReLU. Specifically, the ConvID layer performs convolution operations on a second dimension (feature dimension) of the input tensor to generate 32 feature maps. The ReLU activation function is defined as f(x)=max(0, x), which may introduce non-linear characteristics and accelerate training convergence.

Following the convolutional layer, a pooling layer may be applied, such as a max pooling layer (MaxPooling1D), in which a pool size may be set to 1 or other values. The pooling layer performs dimensionality reduction on the feature maps, retaining the most salient features and reducing the number of parameters. Commonly used pooling methods include max pooling and average pooling. Max pooling selects a maximum value within each pooling window as an output, thus retaining the most salient feature signals.

Following the pooling layer, a second convolutional layer may be further applied. A number of filters and a kernel size of the second convolutional layer may be set according to requirements, and the activation function may also employ ReLU. In some implementations, multiple convolutional and pooling layers may be stacked to increase expressiveness and a degree of feature abstraction of the model. Through stacking of multiple convolutional and pooling layers, the CNN may learn a hierarchical representation from low-level features (such as local patterns) to high-level features (such as abstract concepts) in a layer-by-layer manner.

After processing by the convolutional and pooling layers, the feature maps need to be flattened into a one-dimensional vector for processing by the fully connected layer. The flatten layer reshapes multidimensional feature maps into a one-dimensional vector, having a length equal to a total number of elements in all feature maps. The flattened feature vector is then input into the fully connected layer.

In some implementations, the fully connected layer includes one or more hidden layers and one output layer. For example, a first fully connected layer (dense layer) may include 50 neurons, with an activation function employing ReLU. Each neuron in the fully connected layer is connected to all neurons of a preceding layer and is capable of learning global interactions among features. During training, a Dropout layer may be added between the fully connected layers to prevent overfitting. The Dropout layer randomly sets outputs of a certain proportion of neurons to zero during training, for example, with a Dropout rate set to 0.5, forcing the model to learn more robust feature representations and avoiding excessive dependence on specific neurons.

In a training mode, survival data of multiple training samples may be received to train the deep learning model in a supervised manner. Specifically, the training data includes input features (the residual value and expression values of the immune gene signatures) and corresponding labels (survival time and event indicator). The deep learning model generates predicted risk scores based on the input features and calculates a loss between the predicted values and the true labels.

In some implementations, a Cox proportional hazards loss function is employed to train the CNN model. The Cox loss function is based on a partial likelihood of a Cox proportional hazards model and is capable of handling survival analysis problems involving censored data. Specifically, for N training samples, the samples are ranked by survival time, and for each event time point, a hazard ratio of a sample having an event at the time point relative to all samples in a risk set is calculated, and the partial likelihood is maximized. By minimizing a negative log partial likelihood, the model may be trained to learn feature representations associated with survival outcomes.

In some other implementations, the loss function may employ a Mean Squared Error (MSE), a Cross-Entropy Loss, or other loss functions suitable for survival analysis. Model training employs a backpropagation algorithm and a gradient descent optimizer to update model parameters. In one implementation, an Adam optimizer (Adaptive Moment Estimation optimizer) is employed, which combines advantages of momentum and an adaptive learning rate to accelerate training convergence and improve stability.

In an inference mode, action S180 does not need to receive survival data, but rather generates the prognosis risk score solely based on the input residual value and expression values of the immune gene signatures. Specifically, for the target subject, after processing through actions S110 to S170, the residual value and the immune feature data of the target subject are generated, converted into a tensor, and input into the trained deep learning model, and the model outputs the prognosis risk score of the target subject.

The prognosis risk score may be a continuous numerical value, where a higher value indicates a higher prognosis risk and a lower value indicates a lower prognosis risk. In some implementations, one or more cutoff points may be set based on a distribution of the prognosis risk scores to classify patients into a high-risk group and a low-risk group, or into multiple risk levels. For example, a median of the prognosis risk scores of a training set or a validation set may be used as a cutoff point. Patients having prognosis risk scores higher than the median are classified into the high-risk group, and patients having prognosis risk scores lower than the median are classified into the low-risk group.

For example, assuming that the target subject is a new hepatocellular carcinoma patient, after processing through the aforementioned actions S110 to S170, a residual value ε_{target}=0.4 and an immune feature vector S_{target}=[2.1, 1.5, 0.8, 1.2] (assuming that four immune gene signatures are selected) are generated. Action S180 concatenates these features into a vector [0.4, 2.1, 1.5, 0.8, 1.2], reshapes the vector into a tensor having a shape of (1, 5, 1), and inputs the tensor into the trained CNN model. After convolution, pooling, flattening, fully connected, and other operations, the CNN outputs a prognosis risk score of 0.65. If 0.65 is higher than a median of the prognosis risk scores of the reference population, the patient is classified into the high-risk group, and more aggressive treatment or more intensive follow-up is recommended.

In some implementations, other deep learning models may be employed in place of the CNN. For example, a Recurrent Neural Network (RNN), a Long Short-Term Memory (LSTM) network, a Gated Recurrent Unit (GRU), a Transformer, or a hybrid architecture combining the CNN and the RNN (such as CNN-LSTM) may be employed. In some other implementations, more complex CNN architectures may be employed, such as ResNet, DenseNet, Inception, or other advanced convolutional architectures. Those skilled in the art will understand that different deep learning architectures each have their own characteristics and applicable scenarios, and the selection of an appropriate architecture may depend on data characteristics, computational resources, and performance requirements.

In some implementations, hyperparameter tuning may be performed on the deep learning model to optimize performance. For example, grid search, random search, Bayesian optimization, or other hyperparameter optimization methods may be employed to search for an optimal combination of hyperparameters such as a number of convolutional layers, a number of filters, a kernel size, a number of neurons in the fully connected layer, a learning rate, and a batch size. In some other implementations, an ensemble learning method may be employed to train multiple deep learning models and combine prediction results thereof, so as to improve prediction robustness and accuracy.

In other words, action S180 tensorizes the residual value and the expression values of the multiple immune gene signatures and inputs the tensorized data into the deep learning model to extract non-linear interactions and high-dimensional feature representations between the two, thus generating the prognosis risk score. In some implementations, through the tensor conversion, the residual value and the multidimensional immune feature data may be integrated into a format suitable for processing by the deep learning model. In some implementations, through the CNN architecture, local features and abstract representations may be effectively learned.

In a training mode, by receiving survival data of multiple training samples and employing an appropriate loss function (such as the Cox proportional hazards loss function), the model may be trained to learn features associated with prognosis outcomes. In an inference mode, for a new target subject, the model may generate an individualized prognosis risk score based on the residual value and the immune feature data of the target subject. Advantageously, through the non-linear feature extraction capability of the deep learning model, the method of the present disclosure is able to capture complex immune interaction patterns that cannot be discovered by conventional linear statistical models, and to integrate residual-driven dynamic signals and multidimensional immune features to generate a prognosis risk score having higher prediction accuracy and biological significance, thus providing advantageous support for clinical prognosis assessment and treatment decision-making.

The following describes an application and technical effects of the method of the present disclosure in a model training and validation stage using a specific implementation. This implementation employs the TCGA-LIHC (The Cancer Genome Atlas Liver Hepatocellular Carcinoma) hepatocellular carcinoma dataset to demonstrate how to train the deep learning model using a sample set with known prognosis information and to validate the prognosis risk assessment capability of the model. The trained model may be used for subsequent prognosis risk assessment of new patients. Those skilled in the art will understand that the following implementation is merely illustrative, and the method of the present disclosure may also be applied to other cancer types or other datasets.

In this implementation, the TCGA-LIHC dataset is employed, which includes mRNA expression data and clinical survival data of 230 hepatocellular carcinoma patients. The gene expression data is obtained from the UCSC Xena platform and has been normalized. The clinical survival data includes an overall survival time (OS_time) and an event indicator (event), where an event indicator of 1 indicates that the patient has died, and an event indicator of 0 indicates that the patient is still alive or lost to follow-up. In a model training stage, the 230 samples serve as training samples for establishing time-series statistical model parameters and training the deep learning model. The target immune gene is selected as CCL5, and the immune gene signatures include 12 immune-related genes or gene sets, specifically including CD8 T cells, Th1, B cells, Th2, T cells, NK cells, PRF1, GZMB, Tregs, Macrophage, Granulocyte, and MDSCs.

First, CCL5 observed values of the 230 training samples are obtained, and the 230 training samples are ranked in ascending order according to magnitudes of the observed values to form a pseudo-time series having a length of 230. Next, the CCL5 pseudo-time series is modeled using an ARIMA (5, 1, 0) model to establish the time-series statistical model parameters. After the ARIMA model fitting is completed, a corresponding fitted value is calculated for each position in the pseudo-time series, and a difference between the observed value and the fitted value of each training sample is calculated to generate a residual value for each training sample.

Table 1 shows parameter estimation results of the ARIMA (5, 1, 0) model. As shown in Table 1, estimated values of the five autoregressive parameters (ar.L1 to ar.L5) are −0.7491, −0.5649, −0.4410, −0.3706, and −0.1190, respectively. The first four autoregressive parameters have statistical significance (p-values all less than 0.001), and a p-value of the fifth autoregressive parameter is 0.086, which is close to a significance level of 0.05. An estimated value of a residual variance sigma² is 3.1349, which has high statistical significance (p-value less than 0.001). These results indicate that the CCL5 pseudo-time series has a significant autocorrelation structure, and the ARIMA (5, 1, 0) model is able to effectively capture the linear dynamic trend of the CCL5 observed values.

TABLE 1

| | | Standard | | |
|---|---|---|---|---|
| Parameter | Coefficient | Error | Z-value | P-value |
| ar.L1 | −0.7491 | 0.072 | −10.371 | <0.001 |
| ar.L2 | −0.5649 | 0.083 | −6.829 | <0.001 |
| ar.L3 | −0.4410 | 0.085 | −5.199 | <0.001 |
| ar.L4 | −0.3706 | 0.082 | −4.512 | <0.001 |
| ar.L5 | −0.1190 | 0.069 | −1.719 | 0.086 |
| sigma2 | 3.1349 | 0.317 | 9.901 | <0.001 |

After the residual values of the respective training samples are obtained, expression data of the 230 training samples for the 12 immune gene signatures is extracted as the immune feature data of the respective training samples. Next, Spearman correlation coefficients are calculated among the 12 immune gene signatures (based on the expression values of the 230 training samples), and a hierarchical clustering method is used for grouping. Based on a dendrogram structure, the 12 immune gene signatures are divided into three panels.

Specifically, panel 1 includes cytotoxicity-related signatures such as CD8 T cells, PRF1, GZMB, and Th1. These signatures have high positive correlations with one another and collectively reflect an intensity of a cytotoxic immune response in tumor tissue. Panel 2 includes other lymphocyte-related signatures such as B cells, Th2, T cells, and NK cells. These signatures represent infiltration of other types of immune cells. Panel 3 includes myeloid-derived cell-related signatures such as Granulocyte, Tregs, Macrophage, and MDSCs. These signatures may be associated with immunosuppression or immune regulation.

Next, the residual values of the respective training samples and the immune feature data of the respective panels are separately tensorized, and together with corresponding survival data, are input into the CNN model for training. The CNN model employs the following architecture: a Conv1D layer (number of filters=32, kernel_size=1, activation=ReLU), a MaxPooling1D layer (pool_size=1), a Flatten layer, a Dense layer (number of neurons=50, activation=ReLU), a Dropout layer (rate=0.5), and an output layer (number of neurons=1). Training employs an Adam optimizer with a learning rate set to 0.001, a batch size set to 10, a number of training epochs set to 50, and a loss function employing the Cox proportional hazards loss function.

For each panel, a CNN model is trained separately. After training is completed, the trained model is used to generate prognosis risk scores (CNN features) for the training samples. Next, a Cox proportional hazards model is used to evaluate an association between the CNN features and the survival data, and a hazard ratio (HR), a 95% confidence interval (CI), a Cox regression p-value, and a log-rank test p-value are calculated to evaluate a prognosis stratification capability of the model.

Table 2 shows analysis results of associations between CNN features and survival for different immune panels. As shown in Table 2, the CNN features of panel 1 have the strongest association with survival, with a hazard ratio HR of 0.7324 (95% CI: 0.6101-0.8793), a Cox regression p-value of 0.0008, and a log-rank test p-value of 0.0131. An HR less than 1 indicates that a higher CNN feature value is associated with a lower risk of death, i.e., having a protective effect. A Cox regression p-value less than 0.001 indicates that this association has high statistical significance. Panel 2 has an HR of 0.8714 (95% CI: 0.7362-1.0313), a Cox regression p-value of 0.1093, and a log-rank test p-value of 0.0233. The Cox regression p-value of panel 2 does not reach the significance level of 0.05, but the log-rank test p-value reaches the significance level, indicating that panel 2 has borderline significance. Panel 3 has an HR of 0.8783 (95% CI: 0.6373-1.2103), a Cox regression p-value of 0.4276, and a log-rank test p-value of 0.7894, neither of which reaches statistical significance.

TABLE 2

| Immune | | | Cox p / |
|---|---|---|---|
| Panel | Composition (Examples) | HR (95% CI) | log-rank p |
| Panel 1 | CD8 T cells, PRF1, | 0.7324 | 0.0008/ 0.0131 |
| | GZMB, Th1 | (0.6101-0.8793) | |
| Panel 2 | B cells, Th2, T cells, | 0.8714 | 0.1093/ 0.0233 |
| | NK | (0.7362-1.0313) | |
| Panel 3 | Granulocyte, Tregs, | 0.8783 | 0.4276/ 0.7894 |
| | Macrophage, MDSCs | (0.6373-1.2103) | |

The above results indicate that the CNN features of panel 1 (including cytotoxic immune signatures) have the best prognosis stratification capability. This result is consistent with biological significance, because cytotoxic immune markers such as CD8 T cells, PRF1, and GZMB are closely associated with anti-tumor immune responses, and high expression thereof is typically associated with a better prognosis. By combining the CCL5 residual with the immune features of panel 1 and inputting the combination into the CNN model for training, the method of the present disclosure is able to extract residual-driven cytotoxic immune interactions and generate a model having significant prognosis prediction capability. In contrast, panel 2 exhibits borderline significance, and panel 3 does not reach significance, reflecting different degrees of contribution of different immune panels to prognosis and the heterogeneity of the immune microenvironment.

The results of this implementation validate the technical effects of the method of the present disclosure. First, by modeling the CCL5 pseudo-time series using the ARIMA (5, 1, 0) model, the linear dynamic trend of the CCL5 observed values is successfully captured, and de-trending processing is completed by calculating residuals. Second, through Spearman correlation coefficients and hierarchical clustering, the 12 immune gene signatures are organized into three panels having biological significance, each panel representing a group of functionally related immune features. Third, by integrating residuals and immune features through the CNN model for training, non-linear immune interactions are successfully extracted, and a model having prognosis prediction capability is established. Fourth, the CNN features of panel 1 reach high statistical significance (Cox p=0.0008), indicating that the model trained by the method of the present disclosure is able to effectively improve accuracy of prognosis risk stratification.

After model training and validation are completed, the established model may be applied to clinical inference scenarios. Specifically, when a new hepatocellular carcinoma patient needs to undergo prognosis risk assessment, the following process may be performed: receiving gene expression data of the patient (the target subject), and reference gene expression data of the multiple reference samples (such as the 230 samples from TCGA-LIHC or another established reference sample library); obtaining observed values of the target subject and the multiple reference samples for CCL5; ranking the multiple reference samples according to the reference observed values and positioning the target subject therein according to the target observed value to form a pseudo-time series including the target subject; performing a fitting operation on the pseudo-time series using pre-established ARIMA (5, 1, 0) model parameters and calculating a fitted value at the position of the target subject; calculating the residual value of the target subject; extracting expression values of the target subject for the immune gene signatures in a selected immune panel (such as panel 1); and inputting the residual value and the immune feature data into the trained CNN model to generate the prognosis risk score of the target subject. Through this process, an individualized prognosis risk assessment may be provided for a new patient to support clinical treatment decision-making.

In other words, this implementation demonstrates a complete process and validation results of the method of the present disclosure in a model development stage. By combining the time-series statistical model and the deep learning model, the method of the present disclosure is able to capture both the linear dynamics of immune genes and multi-gene non-linear interactions, and to generate a prognosis risk assessment model having greater statistical significance and biological significance than conventional single-gene median grouping methods. Advantageously, the trained model may be repeatedly applied to prognosis assessment of new patients, providing a reproducible and auditable analytical process that may be applied to different cancer types and different datasets, and supporting clinical prognosis assessment and treatment decision-making.

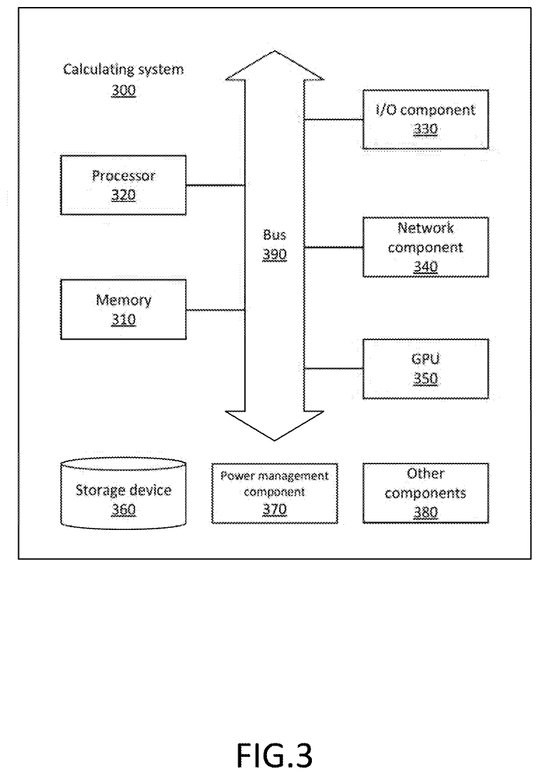

Referring to FIG. 3, FIG. 3 is a schematic block diagram of a computing system according to an implementation of the present disclosure. The cancer prognosis risk assessment method described herein is a computer-implemented method that may be implemented on a computing system 300 having various hardware components. Similarly, the cancer prognosis risk assessment system described herein is also a computer-implemented system that may also be implemented on the computing system 300 having various hardware components.

In some implementations, the computing system 300 may be implemented in a form of an electronic device, including, but not limited to, one or more of the following components: a processor (Central Processing Unit, CPU) 320, a graphics processing unit (GPU) 350, an input/output component 330, a network component 340, a memory 310, a storage device 360, a power management component 370, and other components 380. These components may communicate and transfer data via a bus 390 of the system. However, the present disclosure does not limit specific models, quantities, and configurations of the respective components herein, and those skilled in the art may adjust, select, or add or remove components according to specific requirements and operating environments when implementing the present disclosure.

In some implementations, a primary computing core within the computing system 300 is one or more processors 320. The processor 320 may be responsible for running primary computational processes and related control logic of algorithms such as the time-series statistical model (such as the ARIMA model) and the deep learning model (such as the CNN). In some implementations, the processor 320 is configured to execute processing instructions (i.e., machine-executable instructions) stored in a non-transitory computer-readable medium (such as the storage device 360).

In some implementations, intermediate processing results such as the gene expression data, the fitted data, the residual data, and the immune feature data may be stored in the storage device 360 or the memory 310 as a data transfer medium between respective actions.

In some implementations, in order to improve computational efficiency of the prognosis risk assessment, the computing system 300 may further include one or more graphics processing units 350 specifically designed for performing massive parallel computations. The graphics processing unit 350 is able to effectively improve computational capability of the system when the deep learning model performs feature extraction and training. For example, the graphics processing unit 350 may accelerate computationally intensive operations such as convolution operations, matrix multiplication operations, and gradient descent optimization of the CNN.

In some implementations, the computing system 300 may implement a batch processing mechanism to simultaneously process gene expression data of multiple samples, thus improving efficiency of the prognosis risk assessment. For example, the processor 320 may initiate multiple threads through parallel computing techniques, each thread processing a batch of samples, and utilize the graphics processing unit 350 to accelerate computational processes of ARIMA modeling and CNN training and inference.

In some implementations, the computing system 300 may include various input/output components 330 for receiving user input and displaying system output. For example, the input/output component 330 may include a keyboard, a mouse, a touchpad, a display screen, and other types of sensing devices. Through the input/output component 330, a user may upload gene expression data, set model parameters, view prognosis risk scores, review survival curve charts, and the like.

In some implementations, the computing system 300 may also include a network component 340 for network communication. For example, the network component 340 may include a network interface card for wired or wireless network connections, or a communication module for 3G, 4G, 5G, or other wireless communication technologies. Through the network component 340, the computing system 300 may connect to cloud servers, public databases (such as TCGA, GEO, and the like), or other remote computing resources, so as to obtain gene expression data or perform computationally intensive model training.

In some implementations, the computing system 300 includes one or more memory components 310, such as volatile memory components including a Random Access Memory (RAM). The memory 310 stores program code for executing the cancer prognosis risk assessment method, model parameters, and temporary data during runtime. For example, the memory 310 may store a gene expression data matrix, ARIMA model parameters, CNN model weights, intermediate computation results, and the like.

In some implementations, the computing system 300 may include one or more storage devices 360, such as non-volatile memory components including a Hard Disk Drive (HDD) or a Solid State Drive (SSD). The storage devices 360 may be used to store program code of the cancer prognosis risk assessment system, time-series statistical model parameters, deep learning model parameters, training data, test data, and the like. In addition, the storage devices 360 may also be used to store intermediate results and final outputs such as the gene expression data, the clinical survival data, the fitted data, the residual data, the immune feature data, and the prognosis risk scores.

In some implementations, the computing system 300 may include one or more power management components 370 for providing power to various hardware components of the computing system 300 and managing power consumption thereof. The power management component 370 may include a battery, a power converter, and other power management devices.

In some implementations, the computing system 300 may further include other components 380, such as a cooling fan, a heat dissipator, and various other control and monitoring devices, which are not limited herein by the present disclosure.

Another implementation of the present disclosure provides a computer program product including computer program instructions stored on a computer-readable medium. When a processor of an electronic device executes the computer program instructions, the electronic device is caused to execute the aforementioned cancer prognosis risk assessment method. Specifically, the computer program product includes: program instructions for receiving gene expression data of multiple samples; program instructions for selecting a target immune gene and modeling observed values with a time-series statistical model to generate fitted data; program instructions for calculating a difference between the observed values and the fitted data to generate residual data; program instructions for obtaining expression data of immune gene signatures as immune feature data; and program instructions for inputting the residual data and the immune feature data into a deep learning model to generate a prognosis risk score.

In some implementations, the computer program product may be implemented in a form of computer software. The computer software includes a set of specific program instructions. When the processor executes the set of program instructions, the processor is caused to execute actions of receiving gene expression data, selecting a target immune gene, forming a pseudo-time series, modeling with the ARIMA model, calculating residuals, extracting expression data of immune gene signatures, performing cluster analysis, tensorizing data, inputting data into the CNN model, and generating a prognosis risk score.

In some implementations, the cancer prognosis risk assessment system may be implemented by various hardware architectures. Specifically, the system may be regarded as a functional entity composed of the processor 320, the memory 310, the input/output component 330, the storage device 360, the network component 340, and the like, implementing core functions of receiving gene expression data, modeling with the time-series statistical model, calculating residuals, extracting immune features, and generating a prognosis risk score with the deep learning model.

In detail, the processor 320 is configured to execute program instructions stored in the memory 310. The instructions include: program code instructing the processor 320 to receive gene expression data; program code instructing the processor 320 to select the target immune gene and to initiate the time-series statistical model to model the observed values; program code instructing the processor 320 to calculate the residuals; program code instructing the processor 320 to extract expression data of the immune gene signatures; and program code instructing the processor 320 to initiate and control the deep learning model to perform feature extraction on the residual data and the immune feature data to generate the prognosis risk score.

In some implementations, when the system employs the ARIMA model as the time-series statistical model, the system is able to effectively capture the linear dynamic trend of the target immune gene. The ARIMA model models the observed values based on autoregressive terms, differencing terms, and moving average terms to generate fitted data, enabling the system to decompose the observed values into a linear trend component and a remaining signal component, effectively providing de-trended feature inputs for the subsequent deep learning.

In some implementations, when the system employs the CNN as the deep learning model, the system is able to effectively extract non-linear interactions between the residual and the immune features. The CNN performs local feature extraction and abstract representation learning on the input tensorized data through structures such as convolutional layers, pooling layers, and fully connected layers, and generates a risk score having prognosis prediction capability, enabling the system to provide stable prognosis assessment results when facing a complex immune microenvironment.

It should be noted that the system of the present disclosure is a functional entity composed of various hardware components, including hardware devices such as the processor 320, the memory 310, and the input/output component 330. The time-series statistical model and the deep learning model are stored in the memory 310 or the storage device 360 of the system in the form of program code and parameters, and are loaded and executed by the processor 320. That is, the system does not directly include the time-series statistical model and the deep learning model as hardware components, but rather invokes and runs these models by executing program code stored in the memory 310 to implement the aforementioned method actions. Those skilled in the art will understand that specific technical details of the system implementation have been described in detail in the description of the method in the preceding portion of the present specification. The system implementation and the method implementation of the present disclosure are closely related, and functions of respective modules of the system correspond to respective actions of the method. For example, a module of the system for receiving gene expression data corresponds to action S110, a module for selecting a target immune gene corresponds to action S120, a module for obtaining observed values corresponds to action S130, a module for constructing the pseudo-time series corresponds to action S140, a module for modeling with the time-series statistical model corresponds to action S150, a module for calculating residuals corresponds to action S160, a module for selecting the immune gene signatures corresponds to action S170, and a module for generating the prognosis risk score with the deep learning model corresponds to action S180. Since specific implementations of these actions have been described in detail in the preceding portion of the present specification, those skilled in the art should be able to understand specific implementations of the corresponding system, and therefore redundant description is omitted herein.

In some implementations, a computer-readable medium of the computer program product may be a Read-Only Memory (ROM), a Random Access Memory (RAM), or other suitable computer-readable storage media. The program instructions included in the computer-readable medium may be loaded and executed by one or more processors 320 to implement various functions of the present disclosure. In some implementations, the computer program product may be manufactured in a form of a memory card, a USB flash drive, or other removable storage devices, facilitating transfer and deployment among different electronic devices.

This form of the computer program product enables the method of the present disclosure to be readily implemented on different electronic devices, improving application flexibility and promotion value of the present disclosure. Through the form of the computer program product, a user only needs to install a corresponding program to execute the cancer prognosis risk assessment on any electronic device meeting system requirements, without redeveloping or redesigning a system architecture, significantly reducing a threshold and cost of technical implementation.

In summary, the method of the present disclosure is particularly suitable for clinical scenarios requiring precise prognosis assessment, including but not limited to application fields such as cancer prognosis stratification, treatment decision support, clinical trial risk adjustment, and immunotherapy response prediction. The cancer prognosis risk assessment method and system proposed by the implementations of the present disclosure have multiple technical advantages. First, unlike conventional prognosis assessment methods that only use single-gene median grouping or a small number of clinical variables, the present disclosure is able to simultaneously capture linear dynamics of immune genes and multi-gene non-linear interactions by combining the time-series statistical model and the deep learning model, significantly improving accuracy of prognosis risk stratification. Second, unlike the problem of pure deep learning methods that directly model raw data and are susceptible to trends and noise, the method of the present disclosure performs de-trending processing in advance through the ARIMA model, enabling the deep learning model to focus on learning non-linear immune interaction patterns in the remaining signals, effectively improving prediction performance and robustness of the model. Furthermore, the method of the present disclosure organizes multiple immune gene signatures into panels having biological significance through immune gene cluster analysis, enhancing interpretability of the model, so that prognosis assessment results have not only statistical significance but also biological significance. Finally, the method of the present disclosure provides a reproducible and auditable analytical process applicable to different cancer types and different datasets, providing an effective support tool for clinical prognosis assessment and treatment decision-making.

Based on the above description, it is apparent that various techniques can be configured to implement the concepts described in this application without departing from their scope. Furthermore, although certain implementations have been specifically described and illustrated, those skilled in the art will recognize that variations and modifications can be made in form and detail without departing from the scope of the concepts. Thus, the described implementations are to be considered in all respects as illustrative and not restrictive. Moreover, it should be understood that this application is not limited to the specific implementations described above, but many rearrangements, modifications, and substitutions can be made within the scope of the present disclosure.

What is claimed is:

1. A method for cancer prognosis risk assessment, adapted for assessing a prognosis risk of a target subject, the method comprising:
receiving gene expression data of the target subject and reference gene expression data of a plurality of reference samples;
selecting a target immune gene from the gene expression data and the reference gene expression data;
obtaining a target observed value of the target immune gene from the gene expression data, and obtaining a plurality of reference observed values of the target immune gene from the reference gene expression data;
ranking the plurality of reference samples based on magnitudes of the plurality of reference observed values, and positioning the target subject among the plurality of reference samples based on a magnitude of the target observed value, so as to construct a pseudo-time series;
performing a fitting operation on the pseudo-time series with a time-series statistical model to generate a target fitted value corresponding to a position of the target subject in the pseudo-time series;
calculating a difference between the target observed value and the target fitted value to generate a residual value;
selecting a plurality of immune gene signatures from the gene expression data; and
inputting the residual value and expression values of the plurality of immune gene signatures into a deep learning model to generate a prognosis risk score of the target subject.

2. The method of claim 1, wherein the time-series statistical model is an Autoregressive Integrated Moving Average (ARIMA) model.

3. The method of claim 1, wherein inputting the residual value into the deep learning model comprises: converting the residual value into a tensor format corresponding to an input format of the deep learning model.

4. The method of claim 1, wherein the plurality of immune gene signatures comprises at least one of CD8 T cells, PRF1, GZMB, or Th1.

5. The method of claim 1, wherein selecting the plurality of immune gene signatures comprises:
selecting a plurality of candidate immune gene signatures from the gene expression data;
calculating correlation coefficients among expression values of the plurality of candidate immune gene signatures;
performing a cluster analysis on the plurality of candidate immune gene signatures based on the correlation coefficients; and
selecting the plurality of immune gene signatures based on a result of the cluster analysis.

6. The method of claim 5, wherein the correlation coefficients are Spearman correlation coefficients.

7. A cancer prognosis risk assessment system adapted for assessing a prognosis risk of a target subject, the system comprising:
a memory storing at least one computer-executable instruction; and
a processor coupled to the memory, wherein when the processor executes the at least one computer-executable instruction, the system is caused to:
receive gene expression data of the target subject, and reference gene expression data of a plurality of reference samples;
select a target immune gene from the gene expression data and the reference gene expression data;
obtain a target observed value of the target immune gene from the gene expression data, and obtain a plurality of reference observed values of the target immune gene from the reference gene expression data;
rank the plurality of reference samples based on magnitudes of the plurality of reference observed values, and position the target subject among the plurality of reference samples based on a magnitude of the target observed value, so as to construct a pseudo-time series;
perform a fitting operation on the pseudo-time series with a time-series statistical model to generate a target fitted value corresponding to a position of the target subject in the pseudo-time series;
calculate a difference between the target observed value and the target fitted value to generate a residual value;
select a plurality of immune gene signatures from the gene expression data; and
input the residual value and expression values of the plurality of immune gene signatures into a deep learning model to generate a prognosis risk score of the target subject.

8. The system of claim 7, wherein the time-series statistical model is an Autoregressive Integrated Moving Average (ARIMA) model.

9. The system of claim 7, wherein the system converts the residual value into a tensor format corresponding to an input format of the deep learning model when inputting the residual value into the deep learning model.

10. The system of claim 7, wherein the plurality of immune gene signatures comprises at least one of CD8 T cells, PRF1, GZMB, or Th1.

11. The system of claim 7, wherein, in selecting the plurality of immune gene signatures, the at least one computer-executable instruction, when executed by the processor, further causes the system to:
select a plurality of candidate immune gene signatures from the gene expression data;
calculate correlation coefficients among expression values of the plurality of candidate immune gene signatures;
perform a cluster analysis on the plurality of candidate immune gene signatures based on the correlation coefficients; and
select the plurality of immune gene signatures based on a result of the cluster analysis.

12. The system of claim 11, wherein the correlation coefficients are Spearman correlation coefficients.

13. A non-transitory computer-readable medium storing at least one computer-executable instruction, wherein when the at least one computer-executable instruction is executed by a processor of an electronic device, the electronic device is caused to:
receive gene expression data of a target subject, and reference gene expression data of a plurality of reference samples;
select a target immune gene from the gene expression data and the reference gene expression data;
obtain a target observed value of the target immune gene from the gene expression data, and obtain a plurality of reference observed values of the target immune gene from the reference gene expression data;
rank the plurality of reference samples based on magnitudes of the plurality of reference observed values, and position the target subject among the plurality of reference samples based on a magnitude of the target observed value, so as to construct a pseudo-time series;
perform a fitting operation on the pseudo-time series with a time-series statistical model to generate a target fitted value corresponding to a position of the target subject in the pseudo-time series;
calculate a difference between the target observed value and the target fitted value to generate a residual value;
select a plurality of immune gene signatures from the gene expression data; and
input the residual value and expression values of the plurality of immune gene signatures into a deep learning model to generate a prognosis risk score of the target subject.

14. The non-transitory computer-readable medium of claim 13, wherein the time-series statistical model is an Autoregressive Integrated Moving Average (ARIMA) model.

15. The non-transitory computer-readable medium of claim 13, wherein the electronic device converts the residual value into a tensor format corresponding to an input format of the deep learning model when inputting the residual value into the deep learning model.

16. The non-transitory computer-readable medium of claim 13, wherein the plurality of immune gene signatures comprises at least one of CD8 T cells, PRF1, GZMB, or Th1.

17. The non-transitory computer-readable medium of claim 13, wherein, in selecting the plurality of immune gene signatures, the at least one computer-executable instruction, when executed by the processor, further causes the electronic device to:
select a plurality of candidate immune gene signatures from the gene expression data;
calculate correlation coefficients among expression values of the plurality of candidate immune gene signatures;
perform a cluster analysis on the plurality of candidate immune gene signatures based on the correlation coefficients; and
select the plurality of immune gene signatures based on a result of the cluster analysis.

18. The non-transitory computer-readable medium of claim 17, wherein the correlation coefficients are Spearman correlation coefficients.
